(19) United States
(12) Patent Application Publication
(10) Pub. No.: US 2011/0184477 A1
(43) Pub. Date: Jul. 28, 2011

(54) Aiming Arm for Locking of Bone Nails

(75) Inventors: Alberto A. Fernandez Dell'Oca, Montevideo (UY); Markus Buettler, Oensingen (CH)

(73) Assignee: Synthes Usa LLC

(21) Appl. No.: 12/672,956

(22) PCT Filed: Sep. 11, 2008
(86) PCT No.: PCT/US08/76003

§ 371 Date: Feb. 10, 2010

Related U.S. Application Data
(60) Provisional application No. 60/960,053, filed on Sep. 13, 2007.

(51) Int. Cl. A61B 17/56

(52) U.S. Cl. 606/86 R

(57) ABSTRACT

An aiming arm (4) comprises a rigid distal member a distal portion (4′) of which is configured to be releasably coupled to a proximal end (1″) of an implant (1) to be implanted in a medullary canal of a bone (2), so that, when coupled to the proximal end of an implant, an orientation of the proximal end of the implant relative to the distal portion of the aiming arm remains constant in combination with a rigid proximal member (4″) a distal portion of which is rotatably coupled to a proximal end of the distal member. The proximal member includes an aligning feature (8) defining an axis. (10) aligned with an axis of a fixation element receiving hole (9) extending through a distal portion of the implant, the proximal member being rotatable after implantation of the implant, to an adjusted configuration in which the aligning feature is aligned with a post-implantation orientation of the fixation element.

### BACKGROUND INFORMATION

Bone nails such as intramedullary nails are usually locked at two locations—at a first location close to the entry point and a second location far from the entry point. The end of the nail which is inserted into the bone and penetrates most deeply from the entry site is identified as the distal end while the end of the nail that remains adjacent to the entry site is referred to as the proximal end. As used in this application, the term distal refers to a direction away from an insertion point of an intramedullary implant (i.e., the leading end which is first inserted into the bone is the distal end regardless of the end of the bone into which this leading end is inserted) while the term proximal refers to the opposite direction. The locking of such nails is currently done using either mechanical aiming instruments (e.g., aiming arms) or X-ray guidance.

Mechanical aiming instruments such as those disclosed in U.S. Pat. No. 6,514,253 are generally removably attached to the proximal end of the nail and may provide concentric alignment with proximal screw holes to enable reliable drilling.

An advantage of this type of mechanical aiming arm is that neither the patient nor the surgeon will be exposed to X-rays. However, even when the aiming arm ensures accurate insertion of the proximal end of the nail, distal screw holes may not be properly aligned if the nail is deformed while being driven into the bone.

X-ray guidance is what is used most often for distal locking except when the nail involved is very short. The procedure starts by precisely positioning an X-ray beam along an axis of one or more of the nail holes. However, this may be difficult for the X-ray technician. The nail casts a dark, elongate image on the X-ray monitor, while the nail holes appear as light circles or ovals. In particular, the nail holes will appear circular only when, through a complex 3D procedure, the X-ray source is positioned with the X-ray beam parallel to the axis of the nail hole.

Various aiming guides are already known n the art to be used in conjunction with the X-ray source to accurately place the locking bone screws across both a fractured bone and an implanted intramedullary nail, such as the one disclosed in U.S. Pat. No. 4,88,535.

All these X-ray guided procedures require that the X-ray source be positioned with the X-ray beam parallel to the axis of the nail hole. This is often far from simple and sometimes is not even possible. It may also undesirably increase exposure of the surgeon, patient and operating room staff to X-rays while lengthening the procedure.

As disclosed in U.S. Published Appln. No. 2006/0106400, an aiming arm with radiopaque markers may be used in conjunction with an X-ray source positioned non-parallel to the axis of the nail hole. However, learning to use this device has proved difficult.

### SUMMARY OF THE INVENTION

The present invention relates to a novel apparatus and method for locking intramedullary implant that facilitates the targeting and installation of screws thereinto accurately and reliably.

It is therefore an object of the present invention to provide an aiming arm capable of being adjusted to compensate for distortion of an implant such as an intramedullary nail during insertion into the bone making use of snap shots from an X-ray image intensifier.

Further, it is an object of the present invention to reduce exposure of the surgeon(s), patient and operating room staff to X-rays.

The present invention provides an easy and straightforward procedure for the X-ray technician and the surgeon and makes fixation of the distal ends of such an implant simple and fast, thereby addressing one of the most important issues in surgery—shortening the time required to perform the procedure.

The aiming arm of the present invention overcomes the disadvantages of conventional aiming arms by providing an easily obtainable X-ay guidance for distal locking without requiring that the X-ray beam be coaxial with the nail hole, thus reducing the X-ray exposure of the participants in the procedure.

The preferred embodiment of the present invention provides an adjustable aiming arm fastened to a bone nail. The aiming arm is constructed of a radiolucent material with coplanar transverse holes or apertures.

A hollow insert (either radiolucent or not) perfectly fits into the coplanar transverse holes or apertures existing in the radiolucent aiming arm. A wire can be slid inside this hollow insert until the sharp end of the wire touches the bone being fixed.

The image shown by a single X-ray snapshot in this position gives the surgeon precise information as to the amount of nail distortion after insertion into the bone, allowing the physician to determine any required adjustment of the aiming arm adjustment required to compensate for this distortion. Once the aiming arm has been accurately oriented over the nail hole with the aiming arm transverse holes coaxial with the nail holes, the surrounding bone material may be drilled. After the bone has been drilled, locking bone screws are screwed through the protective sleeves previously inserted into the aiming arm transverse holes to fix the distal portion of the nail in a desired location.

The present invention is directed to an aiming arm for placing an implant in a medullary canal of a bone, comprising a rigid distal member a distal portion of which is configured to be releasably coupled to a proximal end of an implant to be implanted in a medullary canal of a bone, so that, when coupled to the proximal end of an implant an orientation of the proximal end of the implant relative to the distal portion of the aiming arm remains constant in combination with a rigid proximal member a distal portion of which is rotatably coupled to a proximal end of the distal member, the proximal member including an aligning feature which, when in an initial configuration, defines an axis aligned with an axis of a fixation element receiving hole extending through a distal portion of the implant transverse to a longitudinal axis of the implant, the proximal member being rotatable after implantation of the implant to an adjusted configuration in which the aligning feature is aligned with a post-implantation orientation of the fixation element.

The present invention is further directed to a method for implanting an implant in a medullary canal of a bone, comprising coupling to a proximal end of an implant to be implanted in a medullary canal a distal member of an aiming arm, the aiming arm including a proximal member a distal portion of which is rotatably coupled to a proximal end of the distal member so that, in an initial configuration, an aligning feature of the proximal member of the aiming arm is aligned with an axis of a fixation element receiving hole extending transversely through a distal portion of the implant and inserting the implant to a desired position within a medullary canal of a bone in combination with imaging the distal portion of the implant including the fixation element receiving hole and aligning feature of the proximal member and rotating the proximal member relative td the distal member into an aligned configuration in which the aligning feature is aligned with the axis of the fixation element receiving hole.

Other objects, advantages and novel features of the present invention will become apparent from the following detailed description of the invention when considered in conjunction with the accompanying drawings.

### BRIEF DESCRIPTION OF THE DRAWINGS

FIG. 1 shows a perspective view of an aiming arm according to the present invention;

FIG. 2 shows a perspective view of the aiming arm of FIG. 1 without showing the surrounding bone and with an image intensifier in position so that an X-ray beam is out of alignment an axis of a transverse distal nail hole;

FIG. 3 shows an image intensifier aspect of the nail and nail hole of FIG. 2 with the X-ray beam out of alignment with the nail hole;

FIG. 4 shows an image intensifier aspect of the nail and nail hole with the device of FIG. 1 in place;

FIG. 5 shows an image intensifier aspect of the nail and nail hole with the device of FIG. 1 in place making use of a line on a transparent sheet to assess an amount of nail deformation before compensation for the deformation;

FIG. 6 shows an image intensifier aspect of the nail and nail hole with the device of FIG. 1 in place making use of the line on the transparent sheet to assess the amount of nail deformation after compensation for the deformation of the nail has been done; and

FIG. 7 shows an image intensifier aspect of the nail and nail hole with the device of FIG. 1 in place making use of a mouse/joystick/keys controlled line, to assess an amount of nail deformation before compensation for the deformation.

### DETAILED DESCRIPTION

The present invention may be further understood with reference to the following description and the appended drawings, wherein like elements are referred to with the same reference numerals. The present invention relates generally to methods and devices for the stabilization and fixation of fractured bones and bone fragments. Specifically, the present invention relates to methods and devices for the stabilization and/or fixation of long bones through the insertion of a stabilizing member longitudinally thereinto. For example, the present invention relates to the placement and fixation of an intramedullary nail within the medullary canal of a long bone such as the femur, humerus, tibia, etc. However, those skilled in the art will understand that the present invention may be employed in stabilizing any long bone through the insertion into a medullary canal thereof of an intramedullary member. Thus, the discussion of this invention in regard to the stabilization of a femur with an antegrade approach is illustrative only.

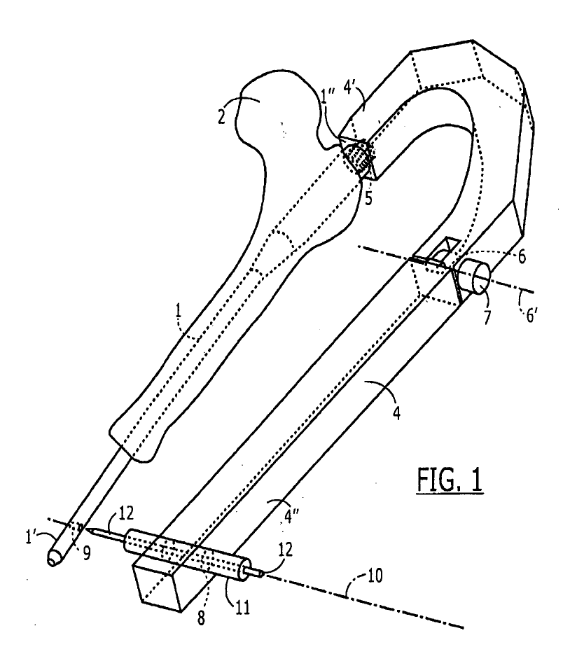

As shown in FIG. 1, a nail 1 is inserted into the medullary canal of a long bone (e.g., a femur 2) until a proximal end of the nail 1 is substantially flush with or within the bone. In this position, the distal portion 1′ of the nail 1 is located deep within the femur 2 with a transverse hole 9 therein positioned to receive a fixation element (e.g., a bone screw) passed laterally through the bone along an axis 10 of the hole 9. The distal portion 4′ of the aiming arm 4 is connected to the proximal end 1″ of the nail 1 by, for example, a set screw 5. Those skilled in the art will understand that the set screw 5 may be replaced by any other mechanism suitable to releasably fix the nail 1 and the distal portion 4′ of the aiming arm 4 to one another in the desired orientation. The distal portion 4′ of the aiming arm 4 is coupled to the proximal portion 4″ thereof via a hinge 6 defining a hinge axis 6′ which allows a user of the aiming arm 4 to compensate for deformation of the nail 1 during insertion as will be described below. A knob 7 locks the hinge 6 in position fixing the proximal portion 4″ in a desired position relative to the distal portion 4′ after a desired degree of compensation for deformation of the nail 1 has been determined as will be described in more detail below. The proximal portion 4″ of the aiming arm 4 has a transverse distal hole 8 extending therethrough which, when the proximal and distal portions 4″, 4′, respectively, have been properly positioned relative to one another, extends along the axis 10. In this position a sleeve 11 passed through the hole 8 is aligned with the axis 10 of the hole 9 of the nail 1. As would be understood by those skilled in the art, the sleeve 11 is preferably closely fitted into the hole 8 so that a lumen of the sleeve 11 remains precisely aligned with the axis of the hole 8 and, consequently, with the axis 10 of the hole 9. A pointer member such as a wire or a pin 12 is then slid through the sleeve 11 to contact the femur 2 at a point at which the axis 10 passes through the surface of the femur 2. As would be understood by those skilled in the art, the pin 12 includes a pointed distal tip which, when the pin is inserted through the sleeve 11, is aligned with the axis of the sleeve 11 and the hole 8.

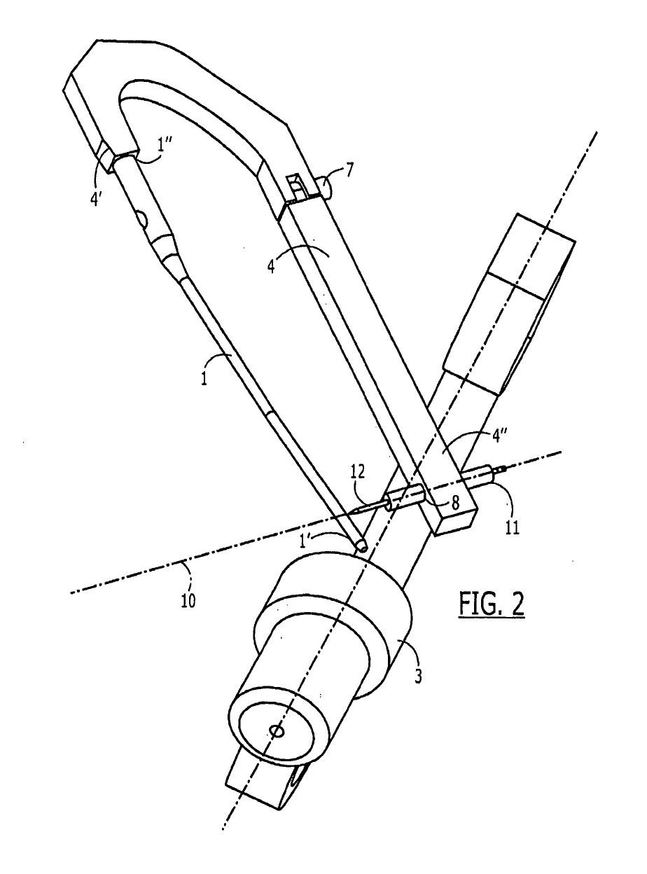

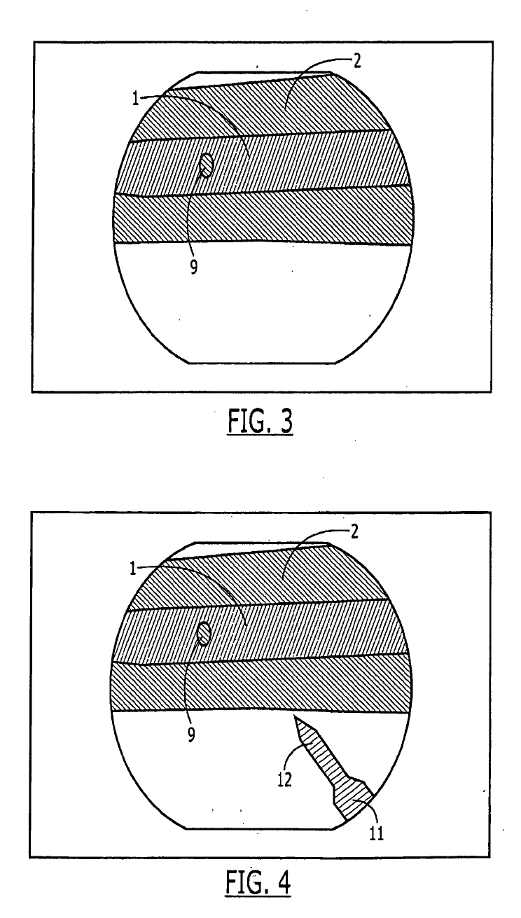

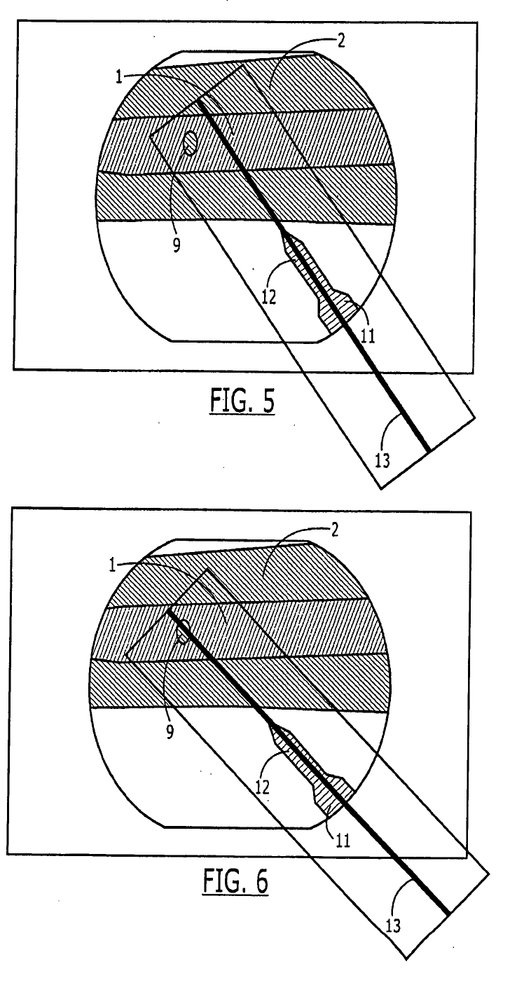

As shown in FIG. 2, the aiming arm 4 is connected to the nail 1 in an orientation, for example, which would have placed the axis of the hole 8 in alignment with the axis 10 of the hole 9 of the nail 1. For example, before insertion into the body, the aiming arm 4 may be coupled to the nail 1 and the orientation of the proximal portion 4″ relative to the distal portion 4′ may be adjusted until the axes of the holes 8 and 9 are perfectly aligned with one another. The relative positions of the proximal and distal portions 4″, 4′, respectively of the aiming arm 4, are then locked using the knob 7 so that the aiming arm 4 may be removed from the nail 1 as needed during the insertion procedure and reattached without altering the alignment of the axes of the holes 8 and 9. However, as described below, deformation of the nail 1 during insertion into the femur 2 (not shown in this Fig.) has moved the hole 9 and the axis 10 out of alignment with the hole 8. The sleeve 11 has been inserted into the hole 8 in the proximal portion 4″ with the pin 12 seated inside the sleeve 11. An X-ray image intensifier 3 is in position but does not need to be in alignment with the axis 10 of the hole 9 extending through the distal portion 1′ of the nail 1. As shown in FIG. 3, an X-ray image produced using the X-ray image intensifier 3 shows the hole 9 as a light spot within the shadow of the nail 1 which is surrounded by the image of the femur 2. The non-circular shape of the hole 9 indicates that the X-ray image intensifier 3 is not aligned with the axis 10 of the hole 9 (i.e., the shape of the hole 9 is foreshortened due to the angle at which the X-rays pass through the femur 2 and the nail 1 relative to the axis 10. FIG. 4 shows substantially the same image as FIG. 3 except that the pin 12 has been extended from the sleeve 11 and is now visible in the image. FIG. 5 then shows an X-ray image onto which a line 13 has been projected along the axis of the pin 12, sleeve 11 and the hole 8 in the proximal portion 4″ of the aiming arm 4. The line 13 is extended over the image until it has passed across the thickness of the nail 1 beyond the hole 9. According to a first embodiment of the invention, the line 13 is superimposed over the X-ray image (e.g., on a monitor) by placing a transparent sheet over the screen of the monitor with a straight line 13 on the sheet overlaying the axis of the pin 12, the sleeve 11 and the hole 8. As seen clearly in FIG. 5, the line 13 does not pass through the hole 9. This is the result of deformation of the nail 1 during insertion which bent the distal portion 1′ out of its initial orientation relative to the proximal portion 1″.

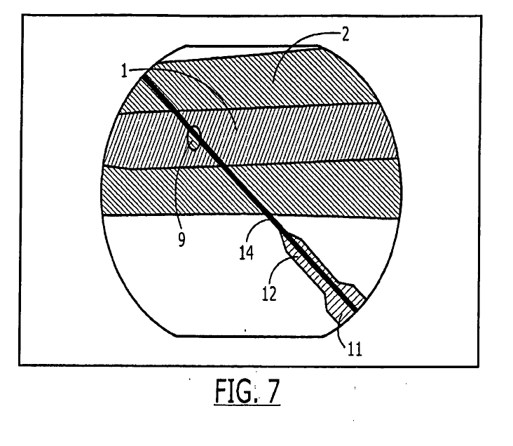

As shown in FIG. 6, the knob 7 may then be loosened and the proximal portion 4″ of the aiming arm 4 may be repositioned relative to the distal portion 4′ until the line 13 on the X-ray image is moved to pass through the center of the hole 9 in the image. The knob 7 may then be retightened to lock the distal and proximal portions 4′, 4″ relative to one another and in this position, the axis of the sleeve 11, the pin 12 and the hole 8 are aligned with the axis 10 of the hole 9 in the distal portion 1′ of the nail 1. The pin 12 may now be replaced by a drill to drill along the axis of the sleeve 11 and the hole 8 so that the hole will pass through the femur 2 along the axis 10 to open into the hole 9.

FIG. 7 shows an X-ray image similar to that of FIG. 6 after the orientation of the proximal and distal portions 4″, 4′ of the aiming arm 4 have been adjusted relative to one another (i.e., by rotation about the hinge 6) to align the axis of the sleeve 11 and the hole 8 with the axis 10 of the hole 9 of the nail 1. Similarly to the image of FIG. 6, the image of FIG. 7 shows the nail 1, the femur 2 and the nail hole 9 along with the tip of the sleeve 11 and the pin 12. In contrast to the apparatus of FIG. 6, however, the embodiment of FIG. 7 the line 14 extending along the axis of the sleeve 11, the hole 8 and the pin 12 is computer generated. Similarly to the line 13 of FIG. 6, the line 14 of FIG. 7 goes straight through the nail hole 9 along the axis 10 thereof. In the same manner described above, after this adjustment of the proximal and distal portions 4″, 4′, respectively, of the aiming arm 4 which align the axis of the hole 8 with the axis 10 of the hole 9 in the nail 1, the pin 12 may be replaced by a drill which may be operated to drill a hole into the bone along the axis 10 to the hole 9.

It will be apparent to those skilled in the art that various modifications and variations may be made in the structure and the methodology of the present invention, without departing from the spirit or scope of the invention. Thus, it is intended that the present invention cover modifications and variations of the invention provided that they come within the scope of the appended claims and their equivalents.

What is claimed is:

1. An aiming arm for implanting an implant in a medullary canal of a bone, comprising:
a rigid distal member a distal portion of which is configured to be releasably coupled to a proximal end of an implant to be implanted in a medullary canal of a bone, so that, when coupled to the proximal end of an implant, an orientation of the proximal end of the implant relative to the distal portion of the aiming arm remains constant; and
a rigid proximal member a distal portion of which is rotatably coupled to a proximal end of the distal member, the proximal member including an aligning feature which, when in an initial configuration, defines an axis aligned with an axis of a fixation element receiving hole extending through a distal portion of the implant transverse to a longitudinal axis of the implant, the proximal member being rotatable after implantation of the implant, to an adjusted configuration in which the aligning feature is aligned with a post-implantation orientation of the fixation element.

2. The aiming arm of claim 1, wherein the proximal member is coupled to the distal member via a hinge, the aiming arm further comprising a locking mechanism for locking the hinge to selectively prevent rotation of the distal and proximal members relative to one another

3. The aiming arm of claim 1, wherein the aligning feature is formed as an aligning hole extending through the proximal member, the defining an axis.

4. The aiming arm of claim 3, wherein the aligning hole is sized to receive therethrough a bone drill so that a drill inserted therethrough drills a hole through the bone aligned with the axis of the fixation element receiving hole.

5. The aiming arm of claim 3, further comprising a sleeve insertable through the aligning hole, the sleeve including a lumen extending therethrough and defining an axis colinear with an axis of the aligning hole.

6. The aiming arm of claim 5, further comprising a pointer member sized to be slidably received within the lumen of the sleeve with a pointed tip of the pointer member aligned with the axis of the sleeve.

7. The aiming arm of claim 1, wherein the distal portion of the distal member is adapted to couple to the proximal end of an intramedullary nail.

8. A method for implanting an implant in a medullary canal of a bone, comprising:
coupling to a proximal end of an implant to be implanted in a medullary canal a distal member of an aiming arm, the aiming arm including a proximal member a distal portion of which is rotatably coupled to a proximal end of the distal member so that, in an initial configuration, an aligning feature of the proximal member of the aiming arm is aligned with an axis of a fixation element receiving hole extending transversely through a distal portion of the implant;
inserting the implant to a desired position within a medullary canal of a bone;
imaging the distal portion of the implant including the fixation element receiving hole and aligning feature of the proximal member;
rotating the proximal member relative to the distal member into an aligned configuration in which the aligning feature is aligned with the axis of the fixation element receiving hole.

9. The method of claim 8, further comprising locking proximal and distal members in the aligned configuration.

10. The method of claim 9, wherein the aligning feature is an aligning hole sized to slidably receive therethrough a drill so that, when in the aligned configuration, a drill inserted therethrough drills a hole through the bone along the axis of the fixation element receiving hole.

11. The method of claim 8, further comprising projecting onto the image of the distal portion of the implant and the aligning feature of the aiming arm a line extending along an axis of the aligning feature to the implant.

12. The method of claim 11, wherein the proximal member is rotated relative to the distal member until the line passes through the image of the fixation element receiving hole.

13. The method of claim 11, wherein the line is generated on a transparent sheet placed over a display showing the image of the distal portion of the implant and the aligning feature of the aiming arm.

14. The method of claim 11, wherein the line is computer generated and inserted into the image of the distal portion of the implant and the aligning feature of the aiming arm.

15. The method of claim 8, wherein the image of the distal portion of the implant and the aligning feature of the aiming arm is generated using an X-ray image intensifier.

16. The method of claim 15, wherein, when generating the image of the distal portion of the implant and the aligning feature of the aiming arm, the X-ray image intensifier is not aligned with the axis of the fixation element receiving hole.

17. The method of claim 10, further comprising the step of inserting through the aligning hole a sleeve including a lumen extending therethrough and inserting into the aligning hole a pointer member having a pointed tip, the pointed tip indicating an axis of the sleeve.

18. The method of claim 17, further comprising removing the pointer member from the sleeve after the aiming arm has been locked in the aligned configuration.
